(19) United States
(12) Patent Application Publication
(10) Pub. No.: US 2007/0174089 A1
(43) Pub. Date: Jul. 26, 2007

(54) SYSTEM AND METHOD FOR GENERATING RELATED PRODUCT RECOMMENDATIONS AND OFFERS

(76) Inventors: Charles C. Koo, Palo Alto, CA (US); Dennis Wu, Palo Alto, CA (US); Roger Bertman, Los Altos, CA (US)

Correspondence Address: SQUIRE, SANDERS & DEMPSEY L.L.P, PATENT DEPARTMENT, ONE MARITIME PLAZA, SUITE 300, SAN FRANCISCO, CA, 94111-3492, US

(21) Appl. No.: 11/627,366

(22) Filed: Jan. 25, 2007

Related U.S. Application Data
(60) Provisional application No. 60/762,792, filed on Jan. 26, 2006.

(51) Int. Cl. G06Q 50/00

(52) U.S. Cl. 705/2

(57) ABSTRACT

A system and method determine a healthcare/personal care product based on symptoms entered. The system and method can then determine related products for recommendation and offers available for the related products and the healthcare/personal care products.

### PRIORITY REFERENCE TO PRIOR APPLICATION

This application claims benefit of and incorporates by reference U.S. Patent Application No. 60/762,792, filed on Jan. 26, 2006, by inventors Charles C. Koo et al. This application also incorporates by reference U.S. patent application Ser. No. 11/315,410 filed on Dec. 22, 2005 and U.S. patent application Ser. No. 11/462,043 filed on Aug. 3, 2006.

### TECHNICAL FIELD

This invention relates generally to healthcare/personal care products (pharmaceuticals, vitamins, over the counter medications, skin care, etc.), and more particularly, but not exclusively, provides a system and method for generating related product recommendations and offers.

### BACKGROUND

A retail store or a pharmacy has tens of thousands of products on the shelf, many of which are complex and require advice from experts. In some cases, consumers do not know the product but only know the problem that they want to solve. For example, there are more than two thousand over-the-counter (OTC) drug products for the treatment of a variety of symptoms and conditions. Consumers entering the store to find the right product to treat their particular symptoms and conditions are faced with a confusing set of choices, and they are ill-equipped and uncertain of how to make the right choice to meet their needs. Faced with this uncertainty, they typically spend a good deal of time, up to 10 to 20 minutes, comparing packages trying to understand the ingredients of each product and how those ingredients relate to the particular symptoms they want to relieve, diseases or conditions they have, or other considerations such as age or allergies. Their intent is to find the right product which has all of the ingredients they need with no ingredients that they don't need or their conditions prohibit. Frequently, after searching on their own, consumers ask a pharmacist for advice. Similar problems exist in most health-and-beauty products such as vitamins, supplements and cosmetics (including skin care products).

From the retailer and/or manufacturers' perspective, marketing as many products as the consumers can bear at the same time has always been a difficult challenge. Manufacturers want to position their products to meet as many consumer symptom needs as possible and to differentiate them from competitors' products with similar effects. Manufacturers spend millions of dollars to package, advertise, and promote their products in their attempt to maximize their share of product sales.

In conjunction with product recommendation, there is also a window of opportunity for cross-selling of related products. Some attempts offer discounts or coupons based on market statistics of purchases in a “basket.” That is they use the general buying pattern among the consumers in each shopping visit (a “basket”) as a proxy to determine additional cross-sell additional products to which the consumer is likely to be receptive. These attempts fail to address the individual consideration of the shopper at the time, especially regarding his/her personal/health factors. Sometimes, pharmacists/specialists try to cross-sell additional products as they converse with the consumer. However, the cross-selling via recommendation and promotion of products can be limited due to the lack of knowledge of the pharmacist and the lack of authorization to provide offers, such as discounts, for the related products.

As such, a new system and method are needed that enable healthcare product cross-selling.

### SUMMARY

Embodiments of the invention enable the recommendation of related products and offering of offers for the related products. “Related products” are cross-selling opportunities provided by a system at retailer store locations. To ensure high relevance, related product lists are generated in the following manner:
The system provides a list of relevant “concepts” used in a kiosk system. These “concepts” can be either explicitly selected by the consumer during an application session or they can be implicitly derived by the system. For example, “Sneezing” (a symptom), “Pregnant women” (a special consideration) and “Infant” (an age group selection) are concepts that can be specified by the consumer. On the other hand, “Allergies” (a disease) is a concept that can be derived by observing a selected “Sneezing” symptom.
The retailer or other entity specifies a list of associated related products against the “target” concepts described above.
Individual stores can optionally apply any “filtering” to mask out certain related products which that they do not carry.

For any given query originated from the kiosk, a knowledge server will utilize the related product data and respond with a list of applicable related products. This list will be sorted by a relevance measure aimed to increase the cross-selling capability.

In an embodiment, the system can provide offers, e.g., print coupons and provide information and/or promotional offers (e.g., a discount) for every consumer to whom products are recommended. Coupons offering discounts can be redeemed with the purchase of the promoted product(s) at specified stores and timeframes which have been agreed by the host retailer and/or the product(s) manufacturer. In an embodiment, the redemption process adheres to standard retailer and/or manufacturer coupon redemption processes, including point of sale (POS) scanning and discounting and manufacturer coupon clearing processes. The product(s) contained on the coupon can be based on the recommended and/or related products. As such, these coupons are highly targeted to consumers based on their specific needs based on their symptoms and conditions and their close proximity in time and space to the point of purchase.

In an embodiment, a method comprises: determining an intent based on a medical symptom; determining a healthcare product based on the determined intent; determining a product related to the healthcare product; and displaying the healthcare product and the related product.

In another embodiment, a method comprises: determining an intent based on a medical symptom; determining a healthcare product based on the determined intent; determining an offer based on the determined healthcare product; and displaying the healthcare product and the offer.

In an embodiment, a system comprises a core, a cross-selling recommendation engine, and a graphical user interface (GUI). The core determines an intent based on a medical symptom and determines a healthcare product based on the determined intent. The recommendation engine determines a product related to the healthcare product. The GUI displays the healthcare product and the related product.

In an embodiment, a system comprises a core, an offer engine, and a GUI. The core determines an intent based on a medical symptom and determining a healthcare product based on the determined intent. The offer engine determines an offer based on the determined healthcare product. The GUI displays the healthcare product and the offer.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a diagram illustrating a kiosk;

FIG. 2 is a block diagram illustrating components of the kiosk of FIG. 1;

FIG. 3 is a block diagram illustrating a persistent memory of the kiosk;

FIG. 4 is a block diagram illustrating an “intent” graph;

FIG. 5 is a table illustrating an example related products database table; and

FIG. 6 is a flowchart illustrating a method of displaying related product recommendations and/or offers.

### DETAILED DESCRIPTION OF THE ILLUSTRATED EMBODIMENTS

The following description is provided to enable any person having ordinary skill in the art to make and use the invention, and is provided in the context of a particular application and its requirements. Various modifications to the embodiments will be readily apparent to those skilled in the art, and the principles defined herein may be applied to other embodiments and applications without departing from the spirit and scope of the invention. Thus, the present invention is not intended to be limited to the embodiments shown, but is to be accorded the widest scope consistent with the principles, features and teachings disclosed herein.

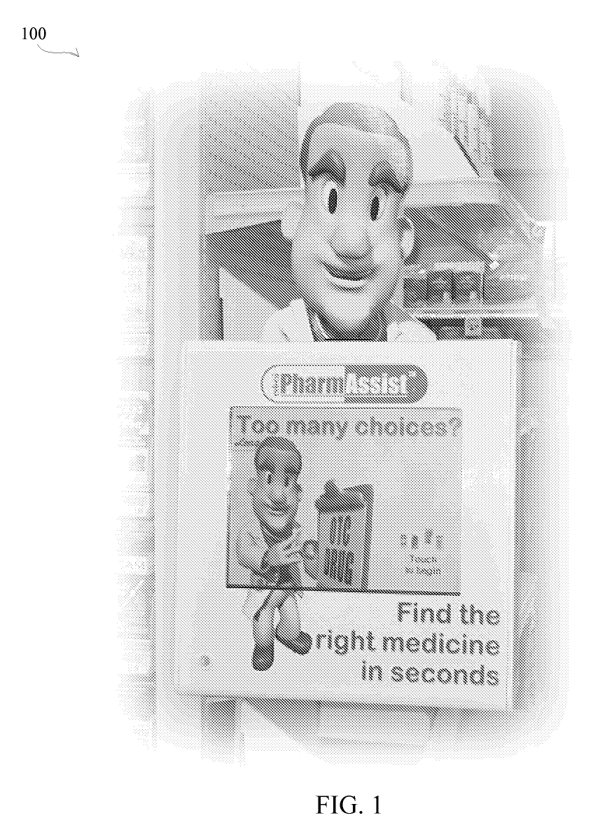

FIG. 1 is a diagram illustrating a kiosk 100. The kiosk 100 implements a process which recommends the correct products to an individual consumer within a retailing/pharmacy environment for the problem (e.g., relieve a set of symptoms and conditions) specified by the consumer. In addition, the kiosk 100 determines related products for cross-selling opportunities and provides offers for the recommended and/or related products.

The kiosk 100 uses search algorithms, e.g., deductive search algorithms, to identify all healthcare products (e.g., OTC drug products) that, in one embodiment, contain the minimum active ingredients to address the symptoms and conditions specified by a consumer. The deductive search algorithms dynamically narrow the universe of potential results as the user specifies symptoms and conditions. Interactions between the search algorithms and a knowledge base recognize symptoms and conditions that are causal or mutually exclusive. As the set of symptoms is specified, other symptoms or conditions that can not coexist in a diagnosis or recommendation are eliminated. Similarly, in one embodiment, as conditions are specified which preclude the use of certain active ingredients, those active ingredients are removed from the list of potential remedies. These algorithms result in a dynamic, real-time identification of possible diseases and treatments. Once the minimal list of active ingredients has been determined through this search algorithm, that list is used to determine all healthcare products that contain those active ingredients. This result meets the criterion of treating all specified symptoms within the constraints of the specified conditions. In an embodiment of the invention, the minimal active ingredients need not considered in product selection.

The final step is to “filter” out all healthcare/personal care products that contain any active ingredients other than those in the minimal list. This step assures that the consumer is recommended only the products which contain only the precise ingredients required to treat the specified symptoms recognizing the specified condition constraints.

Once the set of recommended healthcare products that, in one embodiment, have the minimal set of ingredients and meeting all condition constraints has been determined, the kiosk 100 lists the recommended products. In addition, the kiosk 100 determines related products available for cross-selling and displays them also. The kiosk also determines available offers and displays them with the option of printing the offers (e.g., coupons) or otherwise activating the offers.

In an embodiment of the invention, in addition to or in place of the kiosk 100, a web server can communicate with users coupled to a network and provide the functionality of the kiosk 100 without the need for a user to be physically in a store. For example, the functionality of the kiosk can be incorporated in an online healthcare product store. The kiosk 100 will be discussed in further detail below.

The kiosk 100 is designed to meet a number of criteria which enable it to effectively engage and interact with consumers and to operate efficiently in a retail store environment. These design criteria include the following:
Requires no floor space (“zero footprint”);
Occupies minimal shelf space;
Is fully self-contained (i.e. includes CPU, touch screen, power supply, speaker system, motion detectors, etc., and requires no integration with retailer IT systems);
“Plug and play” installation;
Remote management and support;

In addition to meeting these criteria, the kiosk also has a “Trade Dress” which provides an attractive, engaging presence for the consumer.

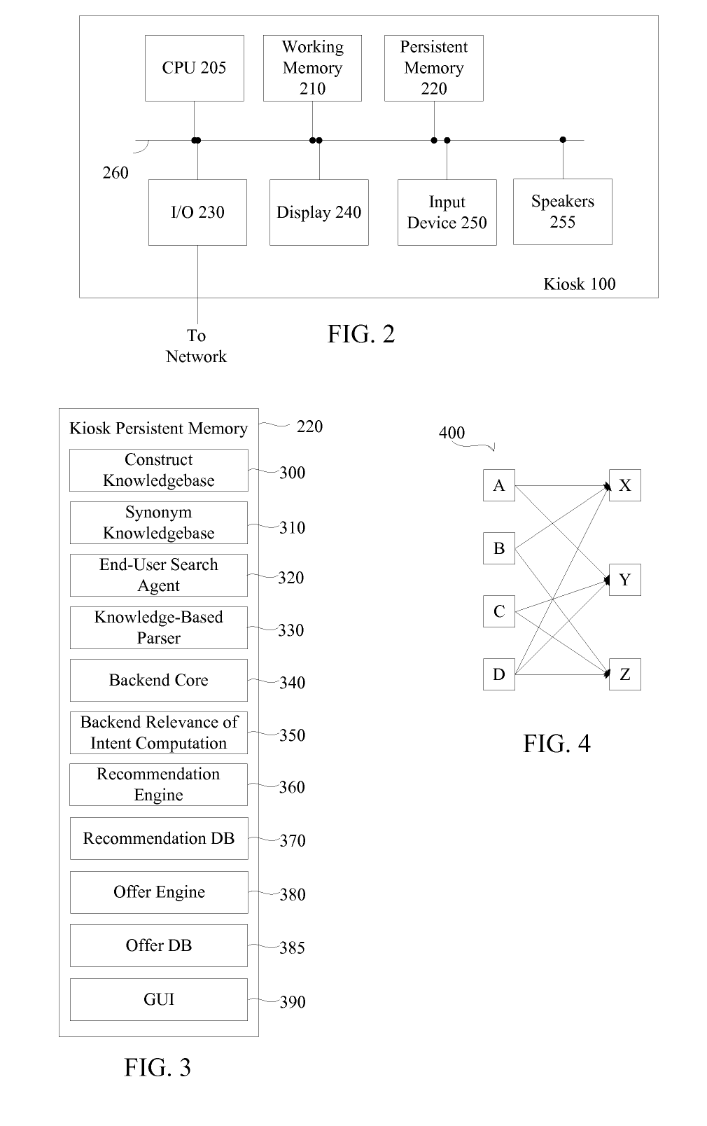

FIG. 2 is a block diagram illustrating components of the kiosk 100. The kiosk 100 includes a central processing unit (CPU) 205; working memory 210; persistent memory 220; input/output (I/O) interface 230; display 240; input device 250; and speakers 255 all communicatively coupled to each other via a bus 260. The CPU 205 may include an INTEL PENTIUM microprocessor, a Motorola POWERPC microprocessor, or any other processor capable to execute software stored in the persistent memory 220. The working memory 210 may include random access memory (RAM) or any other type of read/write memory devices or combination of memory devices. The persistent memory 220 may include a hard drive, read only memory (ROM) or any other type of memory device or combination of memory devices that can retain data after the kiosk 100 is shut off. In an embodiment, the I/O interface 230 is communicatively coupled, via wired or wireless techniques, to a network, such as the Internet. The display 240 may include a flat panel display, cathode ray tube display, or any other display device. The input device 250, may include a keyboard, mouse, touch screen or other device for inputting data, or a combination of devices for inputting data. The speakers 255, which are optional like other components of the invention, emit sound in mono or stereo.

In an embodiment of the invention, the kiosk 100 may also include additional devices, such as network connections, additional memory, additional processors, LANs, input/output lines for transferring information across a hardware channel, the Internet or an intranet, etc. In an embodiment of the invention, the kiosk 100 includes a motion sensor coupled to the bus 260 that activates the display 240 and speakers 255, if any. One skilled in the art will also recognize that the programs and data may be received by and stored in the kiosk 100 in alternative ways. Further, in an embodiment of the invention, an ASIC is used in placed of the kiosk 100.

FIG. 3 is a block diagram illustrating a persistent memory 220 of the kiosk 100. The persistent memory 220 includes a construct knowledgebase 300; a synonym knowledgebase 310; an end-user search agent 320; a knowledge-based parser 330; a backend core 340; a backend relevance of intent computation engine 350; a recommendation engine 360; a recommendation database 370; an offer engine 380; an offer database 385; and a graphical user interface (GUI) 390. Further details are included in Table I, below.

In an embodiment of the invention, the knowledgebase 300 comprises two major categories of knowledge: medical diagnosis and pharmacological knowledge; and OTC medicine knowledge.

All data are integrated together to create a unified internal data structure which can be considered a concept graph (a.k.a. ontology), e.g., see FIG. 4, which are used by the search algorithms to answer end-user queries quickly.

Medical Domain Knowledge contains the following types of information: Symptoms; Diseases/medical conditions; Age group: “Adult”, “Child” and “Infant;” Active ingredients; Special group: risk group; and Side effects

There are also “relationships” built among concepts, an example of which is a comprehensive “causality relationship network” among all symptoms and diseases. This empowers a diagnosis to be determined based on symptoms and conditions.

The OTC medicine knowledge of the knowledgebase 300 contains data for all OTC medicines for the product categories and store environment in which the system is used. The following attributes define an OTC drug: Name; Active ingredient(s); Special considerations; Applicable age group(s); and Side effect(s).

In an embodiment of the invention, the persistent memory 220 can include other or additional software using different algorithms to perform searches for healthcare products. In an embodiment of the invention, searches are not limited to healthcare products.

```
TABLE I
Construct Knowledgebase
  Knowledge structure/construct
    Characteristic mapping (Attributes, taxonomy). For example:
      Concepts: cough
        Is-a: symptom
        ITD: allergy, asthma, COPD, bronchitis
      Concepts: allergy
        Is-a: disease
        DF: cough, wheezing, shortness-of-breath
        ITD: Claritin
      Concepts: Claritin
        Is-a: OTC medicine
        DF: allergy, allergic rhinitis, etc.
Synonym knowledgebase (For example:
  “Shortness of breath” is-a-synonym-of “breathlessness” (strength = 1.0,
which means they mean exactly the same.)
  “Hard to breath” is-a-synonym-of “breathlessness” (strength = 0.8)
End-user search agent (A program)
  UI (auto display of peer terms)
  UI (auto contraction by sets)
  UI (auto expansion for multiple intents/threads)
  UI (auto display of possible diseases)
  interface with the “relevance” count
Knowledge-based Parser (A program)
  map entered words to controlled words
  map controlled words to Concept Constructs based on the synonym
  knowledge base
Backend Core
  The Intent graph (dynamically constructed)
    Connect possible intents (Diagnosis CC)
    Calculate “Relevance Score” of each intent
  Relevance Score Calculation module
    Compute score based on Bayesian network
    Pre-compute scores based on Bayesian network
    Cache and index all possible scores
Backend “relevance” of intent computation
  Bayesian Prior from the counts
  Bayesian Posterior
```

Once the backend core 340 and related software determine relevant products, the GUI 390 displays those products. In addition, the recommendation engine 360 can search the recommendation database 370 for related products to offer. For example, if the disease determined is cold, the recommendation engine 360 will locate Longs Vitamin C in the database 370 and the GUI 390 will display it. Similarly, the offer engine 380 will search the offer database 385 for offers related to the relevant or recommended products and the GUI 390 will display them and enable the offers to be printed or otherwise transmitted to a user (if required). In an embodiment, an offer may be available to all consumers (e.g., a sale) and the purpose of the offer engine 380 is to notify the consumers of the offer to influence purchase of the relevant and/or recommended product.

The GUI 390 attract consumer attention; enables non computer literate consumers to easily interact with the kiosk 100; presents products in strict accordance to the manufacturers' packaging; present products in a manner that is consistent with consumer expectations; and provides complete product advice within seconds.

The GUI 390 employs the following techniques: touch screen interface; motion detection; audio instruction; color graphics; images of product packaging, including “Drug Facts” on all sides of packages; and Virtual Shelf” product presentation.

The touch screen interface enables rapid, intuitive interaction without the use of a keyboard, thereby accommodating consumers who are not versed in using computer keyboards and the presentation of questions in a “multiple choice” fashion, thus minimizing the consumer's role in specifying symptoms and conditions. Motion detection enables the system to attract the consumer's attention by “speaking” as the consumer approaches. Audio instructions compliment the graphic display instructions to accommodate those who are more comfortable with verbal than written communications. The color graphics, particularly full color images of product packages, present a “virtual shelf” experience in which the consumer is presented products on the screen in much the same way they are presented on the shelf, except that only those products that meet their needs are presented. Then, as the consumer touches product images, the product package is “virtually rotated”, again in much the same way a consumer would rotate an actual product package. This experience enables even those consumers who are not versed in using computers to step through the product recommendation and selection process in a manner that is familiar and intuitive.

In an embodiment of the invention, the GUI 390 text descriptions for products will be listed in a random order if the recommended products have no bids for placement by the product's manufacturer, marketer, etc. Recommended products can be listed in a ranked order based on bids as described in U.S. patent application Ser. No. 11/462,043 filed Aug. 3, 2006, which is hereby incorporated by reference. A color image of a product can be displayed if a bid has been placed for it. In an embodiment, product are presented in groups of up to four images at a time, and the order of these groups and the individual images in a group from left to right in which they are presented can be based on the bid amount. The image can include a color image of the face of the product package, plus the ability for the consumer to see images of the other sides of the product. This presentation enables the consumer to read an enlarged image of the drug facts and other information which the bidder has provided on all sides of the package to inform and to convey key messages to the consumer. The consumer views the images by touching the image of the face of the product on the kiosk 100 touch screen 240 and then touching images of subsequent package sides on subsequent screen pages. In an embodiment, the clicking/touching through to see other images of the product can be charged to the bidder (e.g., at 50% of the bid for ranked display). In an embodiment, a video can be shown when a consumer clicks/touches through for an additional fee (e.g., twice the bid fee).

FIG. 4 is a block diagram illustrating an intent graph 400. The graph 400 explains the concept behind the ontological searching method described herein. The graph 400 indicates search terms A, B, C, D and related intents X, Y, and Z. A intends-to-derive (ITD) X or Y; B ITD X or Z; C ITD Y or Z; and D ITD X or Z. The kiosk 100 can then determine peer concepts (search terms) associated with X and Y and display them (e.g., A, B, C, and D). The user's subsequent selection of a peer concept will narrow down the possible intents. For example, the selection of B ITD the intent of X only and the elimination of Y. In an embodiment of the invention, it is possible to have two intents simultaneously (e.g., a person could have symptoms of two different diseases indicating that he/she has two different diseases). In an embodiment of the invention, the intent for symptoms can also be a treatment or over-the-counter medicine for the symptoms, e.g., for the symptom headache, the intent is aspirin.

The “derived from” (DF) relations allow the user to select an intent and conversely narrows the selectable choices of the search terms for the user. The combination and iteration of ITDs and DFs substantially reduce the computation and formulate a refined query, and thus search results rapidly.

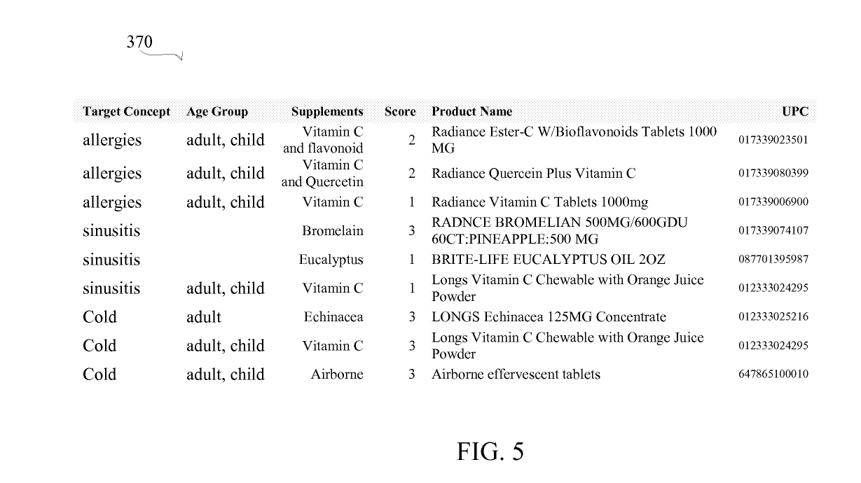

FIG. 5 is a table illustrating an example related products database table 370. The database 370 includes a target concept (e.g., intent, peer concept), optionally age group, optionally product type (e.g., supplement type), optionally score (e.g., relevance to target concept), product name or other identifier, and optionally UPC number. As such, once an intent is determined, a related product can also be recommended. In an embodiment of the invention, the database 370 can be combined with the offer database 385, such that offers for related products would be stored in the database 370.

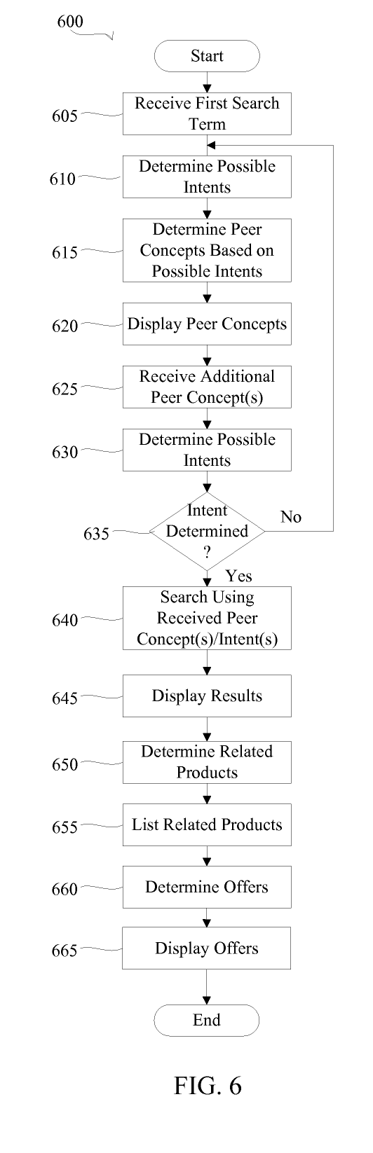

FIG. 6 is a flowchart illustrating a method 600 of displaying related product recommendations and/or offers. In an embodiment of the invention, the kiosk 100 performs the method 600. First, a search term (e.g., symptom) is received (605). Possible intents (disease diagnosis) are then determined (610). Then possible search terms are determined (615) and displayed (620) based on possible intents. A user then selects one or more additional search terms, which are received (625) and possible intents are then determined (630). Due to the receipt of additional search terms, the intent may be determined as discussed above in conjunction with FIG. 5. If the intent is (635) determined or there are no more search terms, then a search is performed (640) based on intent(s) and/or search term(s) selected by the user and received. In an embodiment, the method 600 can include transmitting the search term(s) and/or intent(s) to a search engine to perform the search instead of the performing (640). Otherwise, the method 600 repeats from (610). In an embodiment of the invention, the method 600 can be halted at any point and the search performed (640) using any received search term(s) and/or intent(s). In an embodiment of the invention, the method 600 also includes constraints in the search based on limitations entered by a consumer (e.g., if the consumer indicates an allergy to an antibiotic, any product having that antibiotic will be excluded from search results). Constraints/limitations can be based on allergies, age, dietary restrictions, and/other factors. In an embodiment of the invention, other search methods can be used to determine relevant healthcare products.

Once the search (640) is completed, search results are displayed (645). Related products are then determined (650) be searching a related products database having related products. The search can be based on search terms, intents, peer concepts, and/or search results. The determined related products are then listed (655). Similarly, offers are determined (660) by searching an offer database based on search results and determined related products. The offers are then displayed (665) and the method 600 ends.

In an embodiment of the invention, either the related products determination (650) and display (655) or the offer determination (660) and display (665) are performed. In an embodiment of the invention, the method 600 is performed in an order other than that described above.

The foregoing description of the illustrated embodiments of the present invention is by way of example only, and other variations and modifications of the above-described embodiments and methods are possible in light of the foregoing teaching. For example, while embodiments of the invention are used for the searching and ranking of healthcare products, it can be used for the searching and ranking of anything. Further, any search algorithm can be used. Although the network sites are being described as separate and distinct sites, one skilled in the art will recognize that these sites may be a part of an integral site, may each include portions of multiple sites, or may include combinations of single and multiple sites. Further, components of this invention may be implemented using a programmed general purpose digital computer, using application specific integrated circuits, or using a network of interconnected conventional components and circuits. Connections may be wired, wireless, modem, etc. The embodiments described herein are not intended to be exhaustive or limiting. The present invention is limited only by the following claims.

What is claimed is:

1. A computer-based method, comprising:
determining an intent based on a medical symptom;
determining a healthcare product based on the determined intent;
determining a product related to the healthcare product; and
displaying the healthcare product and the related product.

2. The method of claim 1, wherein the determining the healthcare product is further based on the medical symptom.

3. The method of claim 1, wherein determining the related product is based on the determined healthcare product or personal considerations.

4. The method of claim 1, wherein determining the related product is based on the determined intent.

5. The method of claim 1, wherein determining the related product is based on the medical symptom.

6. The method of claim 1, further comprising:
determining an offer based on the determined related product and the determined medical product; and
displaying the offer.

7. A computer-based method, comprising:
determining an intent based on a medical symptom;
determining a healthcare product based on the determined intent;
determining an offer based on the determined healthcare product; and
displaying the healthcare product and the offer.

8. The method of claim 7, wherein the determining the healthcare product is further based on the medical symptom.

9. A system, comprising:
means for determining an intent based on a medical symptom;
means for determining a healthcare product based on the determined intent;
means for determining a product related to the healthcare product; and
means for displaying the healthcare product and the related product.

10. A system, comprising:
means for determining an intent based on a medical symptom;
means for determining a healthcare product based on the determined intent;
means for determining an offer based on the determined healthcare product; and
means for displaying the healthcare product and the offer.

11. A system, comprising:
a core for determining an intent based on a medical symptom and determining a healthcare product based on the determined intent;
a recommendation engine for determining a product related to the healthcare product; and
a GUI for displaying the healthcare product and the related product.

12. The system of claim 11, wherein the core determines the healthcare product further based on the medical symptom.

13. The system of claim 11, wherein the recommendation engine determines the related product based on the determined healthcare product.

14. The system of claim 11, wherein the recommendation engine determines the related product based on the determined intent.

15. The system of claim 11, wherein the recommendation engine determines the related product based on the medical symptom.

16. The system of claim 11, further comprising:
an offer engine for determining an offer based on the determined related product and the determined medical product; and
where in the GUI further displays the offer.

17. A system, comprising:
a core for determining an intent based on a medical symptom and determining a healthcare product based on the determined intent;
an offer engine for determining an offer based on the determined healthcare product; and
a GUI for displaying the healthcare product and the offer.

18. The system of claim 7, wherein the core further determines the healthcare product is further based on the medical symptom.

19. A computer-readable medium having stored thereon instructions to cause a computer to execute a method, the method comprising:
determining an intent based on a medical symptom;
determining a healthcare product based on the determined intent;
determining a product related to the healthcare product; and
displaying the healthcare product and the related product.

20. A computer-readable medium having stored thereon instructions to cause a computer to execute a method, the method comprising:
determining an intent based on a medical symptom;
determining a healthcare product based on the determined intent;
determining an offer based on the determined healthcare product; and
displaying the healthcare product and the offer.
